(19) United States
(12) Patent Application Publication
(10) Pub. No.: US 2009/0253203 A1
(43) Pub. Date: Oct. 8, 2009

(54) Reprogramming a Cell by Inducing a Pluripotent Gene Through Use of a Small Molecule Modulator

(75) Inventors: Kenneth J. Eilertsen, Baton Rouge, LA (US); Rachel A. Power, Zachary, LA (US)

Correspondence Address: WHYTE HIRSCHBOECK DUDEK S.C.;INTELLECTUAL PROPERTY DEPARTMENT, 33 East Main Street, Suite 300, Madison, WI, 53703-4655, US

(73) Assignee: NuPotential, Inc., Baton Rouge, LA (US)

(21) Appl. No.: 12/419,915

(22) Filed: Apr. 7, 2009

Related U.S. Application Data
(63) Continuation-in-part of application No. 11/497,064, filed on Aug. 1, 2006.
(60) Provisional application No. 60/704,465, filed on Aug. 1, 2005.
(60) Provisional application No. 61/043,066, filed on Apr. 7, 2008.
(60) Provisional application No. 61/042,890, filed on Apr. 7, 2008.
(60) Provisional application No. 61/042,995, filed on Apr. 7, 2008.
(60) Provisional application No. 61/113,971, filed on Nov. 12, 2008.

(51) Int. Cl. C12N 5/06

(52) U.S. Cl. 435/377; 435/325

(57) ABSTRACT

The invention relate to methods, compositions, and kits for reprogramming a cell. In one embodiment, the invention relates to a method comprising inducing the expression of at least one gene that contributes to a cell being pluripotent or multipotent. In yet another embodiment, the method comprises exposing a cell to a small molecule modulator that induces the expression of at least one gene that contributes to a cell being pluripotent or multipotent. In yet another embodiment, the invention relates to a reprogrammed cell and an enriched population of reprogrammed cells that can have characteristics of an ES-like cell can be re- or trans-differentiated into various differentiated cell types.

### CROSS-REFERENCE TO RELATED PATENT APPLICATIONS

This application is a continuation-in-part of U.S. patent application Ser. No. 11/497,064, filed Aug. 1, 2006, which claims benefit under 35 U.S.C. § 119(e) of U.S. Provisional Application 60/704,465, filed Aug. 1, 2005, and also claims benefit under 35 U.S.C. § 119(e) of U.S. Provisional Application 61/043,066, filed Apr. 7, 2008; U.S. Provisional Application 61/042,890, filed Apr. 7, 2008; U.S. Provisional Application 61/042,995, filed on Apr. 7, 2008; and U.S. Provisional Application 61/113,971, filed Nov. 12, 2008, each of which is incorporated herein by reference as if set forth in its entirety.

### FIELD OF THE INVENTION

Embodiments of the invention relate to the fields of cell biology, stem cells, cell differentiation, somatic cell nuclear transfer and cell-based therapeutics. More specifically, embodiments of the invention are related to methods, compositions and kits for reprogramming cells and cell-based therapeutics.

### BACKGROUND OF THE INVENTION

Regenerative medicine holds great promise as a therapy for many human ailments, but also entails some of the most difficult technical challenges encountered in modern scientific research. The technical challenges to regenerative medicine include low cloning efficiency, a short supply of potentially pluripotent tissues, and a generalized lack of knowledge as to how to control cell differentiation and what types of embryonic stem cells can be used for selected therapies. While ES cells have tremendous plasticity, undifferentiated ES cells can form teratomas (benign tumors) containing a mixture of tissue types. In addition, transplantation of ES cells from one source to another likely would require the administration of drugs to prevent rejection of the new cells.

Attempts have been made to identify new avenues for generating stem cells from tissues that are not of fetal origin. One approach involves the manipulation of autologous adult stem cells. The advantage of using autologous adult stem cells for regenerative medicine lies in the fact that they are derived from and returned to the same patient, and are therefore not subject to immune-mediated rejection. The major drawback is that these cells lack the plasticity and pluripotency of ES cells and thus their potential is uncertain. Another approach is aimed at reprogramming somatic cells from adult tissues to create pluripotent ES-like cells. However, this approach has been difficult as each cell type within a multi-cellular organism has a unique epigenetic signature that is thought to become fixed once cells differentiate or exit from the cell cycle.

Cellular DNA generally exists in the form of chromatin, a complex comprising nucleic acid and protein. Indeed, most cellular RNA molecules also exist in the form of nucleoprotein complexes. The nucleoprotein structure of chromatin has been the subject of extensive research, as is known to those of skill in the art. In general, chromosomal DNA is packaged into nucleosomes. A nucleosome comprises a core and a linker. The nucleosome core comprises an octamer of core histones (two each of H2A, H₂B, H3 and H4) around which is wrapped approximately 150 base pairs of chromosomal DNA. In addition, a linker DNA segment of approximately 50 base pairs is associated with linker histone H1. Nucleosomes are organized into a higher-order chromatin fiber and chromatin fibers are organized into chromosomes. See, for example, Wolffe “Chromatin: Structure and Function” 3.sup.rd Ed., Academic Press, San Diego, 1998.

Chromatin structure is not static, but is subject to modification by processes collectively known as chromatin remodeling. Chromatin remodeling can serve, for example, to remove nucleosomes from a region of DNA; to move nucleosomes from one region of DNA to another; to change the spacing between nucleosomes; or to add nucleosomes to a region of DNA in the chromosome. Chromatin remodeling can also result in changes in higher order structure, thereby influencing the balance between transcriptionally active chromatin (open chromatin or euchromatin) and transcriptionally inactive chromatin (closed chromatin or heterochromatin).

Chromosomal proteins are subject to numerous types of chemical modification. One mechanism for the posttranslational modification of these core histones is the reversible acetylation of the epsilon-amino groups of conserved highly basic N-terminal lysine residues. The steady state of histone acetylation is established by the dynamic equilibrium between competing histone acetyltransferase(s) and histone deacetylase(s) herein referred to as HDAC. Histone acetylation and deacetylation has long been linked to transcriptional control. The reversible acetylation of histones can result in chromatin remodeling and as such act as a control mechanism for gene transcription. In general, hyperacetylation of histones facilitates gene expression, whereas histone deacetylation is correlated with transcriptional repression. Histone acetyltransferases were shown to act as transcriptional coactivators, whereas deacetylases were found to belong to transcriptional repression pathways.

The dynamic equilibrium between histone acetylation and deacetylation is essential for normal cell growth. Inhibition of histone deacetylation results in cell cycle arrest, cellular differentiation, apoptosis and reversal of the transformed phenotype.

Another group of proteins involved in the regulation of gene expression are the DNA methyltransferases (DNMTs), which are responsible for the generation of genomic methylation patterns that lead to transcriptional silencing. DNA methylation is central to many mammalian processes including embryonic development, X-inactivation, genomic imprinting, and regulation of gene expression. DNA methylation in mammals is achieved by the transfer of a methyl group from S-adenosyl-methionine to the C5 position of cytosine. This reaction is catalyzed by DNA methyltransferases and is specific to cytosines in CpG dinucleotides. Seventy percent (70%) of all cytosines in CpG dinucleotides in the human genome are methylated and prone to deamination, resulting in a cytosine to thymine transition. This process leads to an overall reduction in the frequency of guanine and cytosine to about 40% of all nucleotides and a further reduction in the frequency of CpG dinucleotides to about a quarter of their expected frequency.

Four active DNA methyltransferases have been identified in mammals. They are named DNMT1, DNMT2, DNMT3A and DNMT3B. In addition, DNMT3L is a protein that is closely related to DNMT3A and DNMT3B structurally and that is critical for DNA methylation, but appears to be inactive on its own. The methylation of cytosines in promoter regions containing CpG islands leads to transcriptional inactivation of the downstream coding sequence in vertebrate cells.

A family of proteins known as methyl-CpG binding proteins (MBD1 to 4) is thought to play an important role in methylation-mediated transcriptional silencing. MeCP2 was the first member of this family to be characterized and contains a methyl-CpG binding domain (MBD) and a transcriptional-repression domain (TRD), which facilitates an interaction with, and targets the Sin3A/HDAC complex to, methylated DNA. Like MeCP2, MBD1, MBD2, and MBD3 have been shown to be potent transcriptional repressors. MBD4 is a DNA glycosylase, which repairs G:T mismatches. Each member of this family, with the exception of MBD3, forms complexes with methylated DNA in mammalian cells, and all but MBD1 and MBD4 have been placed in known chromatin-remodeling complexes. The Mi-2 complex couples DNA methylation to chromatin remodeling and histone deacetylation.

Another group of proteins involved in epigenetic regulation are histone methyltransferases (HMT), which are enzymes, histone-lysine N-methyltransferase and histone-arginine N-methyltransferase that catalyze the transfer of one to three methyl groups from the cofactor S-Adenosyl methionine to lysine and arginine residues of histone proteins. Methylated histones bind DNA more tightly, which inhibits transcription.

The structure of chromatin also can be altered through the activity of macromolecular assemblies known as chromatin remodeling complexes. See, for example, Cairns (1998) Trends Biochem. Sci. 23:20 25; Workman et al. (1998) Ann. Rev. Biochem. 67:545 579; Kingston et al. (1999) Genes Devel. 13:2339 2352 and Murchardt et al. (1999) J. Mol. Biol. 293:185 197. Chromatin remodeling complexes have been implicated in the disruption or reformation of nucleosomal arrays, resulting in modulation of transcription, DNA replication, and DNA repair (Bochar et al. (2000) PNAS USA 97(3): 1038 43). Many of these chromatin remodeling complexes have different subunit compositions, but all rely on ATPase enzymes for remodeling activity. There are also several examples of a requirement for the activity of chromatin remodeling complexes for gene activation in vivo

The development of pluripotent or totipotent cells into a differentiated, specialized phenotype is determined by the particular set of genes expressed during development. Gene expression is mediated directly by sequence-specific binding of gene regulatory proteins that can effect either positive or negative regulation. However, the ability of any of these regulatory proteins to directly mediate gene expression depends, at least in part, on the accessibility of their binding site within the cellular DNA. As discussed above, accessibility of sequences in cellular DNA often depends on the structure of cellular chromatin within which cellular DNA is packaged.

Therefore, it would be useful to identify methods, compositions and kits that can induce the expression of genes required for pluripotency, including methods, compositions, and kits that can inhibit the activity of proteins involved in transcriptional repression.

### BRIEF SUMMARY OF THE INVENTION

The invention relates to methods, compositions and kits for reprogramming a cell. Embodiments of the invention relate to methods comprising inducing the expression of a pluripotent or multipotent gene. In yet another embodiment, the invention further relates to producing a reprogrammed cell. In still yet another embodiment, the invention relates to a method comprises inhibiting the activity of a protein that is involved in transcriptional repression. In yet another embodiment, the invention relates to a method for reprogramming a cell comprising altering the activity, expression or activity and expression of a regulatory protein. The method further comprises inducing the expression of a pluripotent or multipotent gene, and reprogramming the cell.

Embodiments of the invention also relate to methods for reprogramming a cell comprising contacting a cell, a population of cells, a cell culture, a subset of cells from a cell culture, a homogeneous cell culture or a heterogeneous cell culture with an agent that inhibits the activity, expression or activity and expression of a protein involved in transcriptional repression, inducing the expression of a pluripotent or multipotent gene, and reprogramming the cell. The method further comprises re-differentiating the reprogrammed cell. In yet another embodiment, the invention relates to a method for reprogramming a cell comprising exposing a cell to a small molecule modulator that alters the expression, activity or expression and activity of a regulatory protein, inducing the expression of a pluirpotent or mulitpotent gene, and selecting a cell, wherein differentiation potential has been restored to said cell.

An agent that alters the activity, expression or activity and expression of a protein involved in transcriptional repression or a regulatory protein includes but is not limited to a small molecule, small molecule inhibitor and a small molecule activator.

An agent that induces the expression of a pluripotent or multipotent gene includes but is not limited to a small molecule, a small molecule inhibitor and a small molecule inhibitor.

Any protein involved in transcriptional repression can be inhibited by the methods of the invention including but not limited to DNA methyltransferases, histone deacetylases, methyl binding domain proteins, histone methyltransferases, components of the SWI/SNF complex, components of the NuRD complex, and components of the INO80 complex.

In some embodiments, at least one small molecule inhibitor can be used to inhibit the activity of a DNA methyltransferase, a histone deacetylase, a methyl binding domain protein, or a histone methyltransferase. In still yet another embodiment, more than one small molecule inhibitor can be used to inhibit the activity of more than one protein involved in transcriptional repression including but not limited to a DNA methyltransferase, a histone deacetylase, a methyl binding domain protein, or a histone methyltransferase.

In still yet another embodiment, the invention relates to a method comprising contacting a cell with a small molecule inhibitor that inhibits the activity of at least one DNMT; demethylating at least one CpG dinucleotide; inducing the expression of at least one gene that contributes to a cell being pluripotent or multipotent, and reprogramming the cell.

In still another embodiment, the invention relates to a method comprising exposing a cell with a first phenotype to a small molecule modulator that alters the activity, expression, or activity and expression of at least one regulatory protein; comparing the first phenotype of the cell to a phenotype obtained after exposing the cell to a small molecule modulator, and selecting the cell that has been reprogrammed, and is pluripotent or multipotent. In yet another embodiment, the method comprises comparing the genotype of a cell prior to exposing the cell to a small molecule modulator to a genotype of the cell obtained after treatment with a small molecule modulator. In still yet another embodiment, the method comprises comparing the phenotype and genotype of a cell prior to exposing the cell to a small molecule modulator to the phenotype and genotype of the cell after exposing the cell to a small molecule modulator.

In yet another embodiment, the invention relates to a method for reprogramming a cell comprising: exposing a cell to a small molecule modulator that induces expression of a pluripotent or multipotent gene; and selecting a cell, wherein differentiation potential has been restored to said cell. In yet another embodiment, the invention relates to a method for reprogramming a cell comprising: exposing a cell to a small molecule modulator that alters the expression, activity or expression and activity of a regulatory protein, inducing expression of a pluripotent or multipotent gene; and selecting a cell, wherein differentiation potential has been restored to said cell.

In still another embodiment, the method comprises culturing or expanding the selected cell to a population of cells. In yet another embodiment, the method comprises isolating cells using an antibody that binds to a protein coded for by a pluripotent or multipotent gene or an antibody that binds to a multipotent marker or a pluripotent marker, including but not limited to SSEA3, SSEA4, Tra-1-60, and Tra-1-81. In still another embodiment, the invention further comprises comparing chromatin structure of a pluripotent or multipotent gene prior to exposure to said small molecule modulator to the chromatin structure obtained after exposure to said small molecule modulator. Cells may also be isolated using any method efficient for isolating cells including but not limited to a fluorescent cell activated sorter, immunohistochemistry, and ELISA. In another embodiment, the method comprises selecting a cell that has a less differentiated state than the original cell.

In another embodiment, the invention relates to a method for reprogramming a cell comprising: exposing a cell with a first transcriptional pattern to a small molecule modulator that induces expression of a pluripotent or multipotent gene; comparing the first transcriptional pattern of the cell to a transcriptional pattern obtained after exposure to said modulator; and selecting a cell, wherein differentiation potential has been restored to said cell. In another embodiment, selecting a cell comprises selecting a cell that has a less differentiated state than the original cell.

In still another embodiment, selecting a cell comprises identifying a cell with a transcriptional pattern that is at least 5-10%, 10-20%, 20-30%, 30-40%, 40-50%, 50-60%, 60-70%, 70-80%, 80-90%, 90-94%, 95%, or 95-99% similar to an analyzed transcriptional pattern of an embryonic stem cell. The entire transcriptional pattern of an embryonic stem cell need not be compared, although it may. Instead, a subset of embryonic genes may be compared including but not limited to 1-5, 5-10, 10-25, 25-50, 50-100, 100-200, 200-500, 500-1,000, 1,000-2,000, 2,000-2,500, 2,500-5,000, 5,000-10,000 and greater than 10,000 genes. The transcriptional patterns may be compared in a binary fashion, i.e., the comparison is made to determine if the gene is transcribed or not. In another embodiment, the rate and/or extent of transcription for each gene or a subset of genes may be compared. Transcriptional patterns can be determined using any methods known in the art including but not limited to RT-PCR, quantitative PCR, a microarray, southern blot and hybridization.

In yet another embodiment, the invention relates to a method for reprogramming a cell comprising: exposing a cell to a small molecule modulator that interferes with the activity, expression, or activity and expression of a first regulatory protein; exposing said cell to a second agent that inhibits the activity, expression or expression and activity of a second regulatory protein, wherein said second regulatory protein has a distinct function from the first regulatory protein, inducing expression of a pluripotent or multipotent gene, and selecting a cell, wherein differentiation potential has been restored to said cell. In another embodiment, the cell or population of cells may be exposed to the first and second agents simultaneously or sequentially. The second agent includes but is not limited to a small molecule, a small molecule inhibitor, a small molecule activator, a nucleic acid sequence, and an shRNA construct.

Embodiments of the invention also include methods for treating a variety of diseases using a reprogrammed cell produced according to the methods disclosed herein. In yet another embodiment, the invention also relates to therapeutic uses for reprogrammed cells and reprogrammed cells that have been re-differentiated. Embodiments of the invention also relate to a reprogrammed cell produced by the methods of the invention. The reprogrammed cell can be re-differentiated into a single lineage or more than one lineage. The reprogrammed cell can be multipotent or pluripotent.

In yet another embodiment, the invention relates to an enriched population of reprogrammed cells produced according to a method comprising the steps of: exposing a cell to a small molecule modulator that induces expression of a pluripotent or multipotent gene; and selecting a cell, wherein differentiation potential has been restored to said cell, and culturing said selected cell to produce population of cells. In still another embodiment, the reprogrammed cell expresses a cell surface marker selected from the group consisting of: SSEA3, SSEA4, Tra-1-60, and Tra-1-81. In yet another embodiment, the reprogrammed cells account for at least 5-10%, 10-20%, 20-30%, 30-40%, 40-50%, 50-60%, 60-70%, 70-80%, 80-90%, 90-95%, 96-98%; or at least 99% of the enriched population of cells.

Embodiments of the invention also relate to kits for preparing the methods and compositions of the invention. The kit can be used for, among other things, reprogramming a cell and generating ES-like and stem cell-like cells.

### BRIEF DESCRIPTION OF THE DRAWINGS

FIG. 1 is a bar graph reporting the up-regulation of Oct-4 and Nanog in primary human lung fibroblasts treated with a DNMT inhibitor (500 μM RG108). MC is the control medium.

FIG. 2 is a bar graph reporting as increase in expression of several pluripotent genes in the presence of VPA in several cell types. HDFa means adult human dermal fibroblasts; HDFf means fetal human dermal fibroblasts; HDFn means neonatal human dermal fibroblasts; and BJF means BJ fibroblasts (foreskin).

FIG. 3 is a bar graph reporting the effects on expression of HDAC11 and HDAC9 in adult and fetal human dermal fibroblasts treated with VPA.

FIG. 4 is a bar graph reporting an increase in expression of Oct-4 in adult human dermal fibroblasts treated with nicotinamide for four days.

FIG. 5 is a bar graph reporting an increase in expression of Oct-4 in adult human dermal fibroblasts treated with sodium phenylbutyrate for four days.

FIG. 6 is a bar graph reporting an increase in expression of Oct-4 in adult human dermal fibroblasts treated with valproxam for four days.

FIG. 7 is a bar graph reporting an increase in expression of Oct-4 in BJ fibroblasts treated with 2-PCPA (histone/lysine 1 demethylase inhibitor) for eight days.

FIG. 8A is a photograph of adult human dermal fibroblasts in fibroblast growth medium.

FIG. 8B is a photograph of adult human dermal fibroblasts in DMEM/F12 medium.

FIG. 8C is a photograph of VPA treated (500 μM) adult human dermal fibroblasts in mTeSR hES cell medium on matrigel.

FIG. 8D is a photograph of VPA treated (500 μM) adult human dermal fibroblasts in mTeSR hES cell medium on matrigel.

### DETAILED DESCRIPTION OF THE PREFERRED EMBODIMENT

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, melt index, temperature etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

“Cell” or “cells,” unless specifically limited to the contrary, includes any somatic cell, embryonic stem (ES) cell, adult stem cell, an organ specific stem cell, nuclear transfer (NT) units, and stem-like cells. The cell or cells can be obtained from any organ or tissue. The cell or cells can be human or other animal. For example, a cell can be mouse, guinea pig, rat, cattle, horses, pigs, sheep, goats, etc. A cell also can be from non-human primates.

“Culture Medium” or “Growth Medium” means a suitable medium capable of supporting growth of cells.

“Differentiation” means the process by which cells become structurally and functionally specialized during embryonic development.

“DNA methyltransferase inhibitor” and “inhibitor of DNA methyltransferase” mean a compound that is capable of interacting with a DNA methyltransferase and inhibiting its activity. “Inhibiting DNA methyltransferase activity” means reducing the ability of a DNA methyltransferase to methylate a particular substrate, such as a CpG dinucleotide sequence. In some embodiments, such reduction of DNA methyltransferase activity is at least about 25% at least about 50%, in other embodiments at least about 75%, and still in other embodiments at least about 90%. In yet another embodiment, DNA methyltransferase activity is reduced by at least 95% and in another embodiment by at least 99%.

“Epigenetics” means the state of DNA with respect to heritable changes in function without a change in the nucleotide sequence. Epigenetic changes can be caused by modification of the DNA, such as by methylation and demethylation, without any change in the nucleotide sequence of the DNA.

“Histone” means a class of protein molecules found in chromosomes responsible for compacting DNA enough so that it will fit within a nucleus.

“Knock down” means to suppress the expression of a gene in a gene-specific fashion. A cell that has one or more genes “knocked down,” is referred to as a knock-down organism or simply a “knock-down.” “Pluripotent” means capable of differentiating into cell types of the 3 germ layers or primary tissue types.

“Pluripotent gene” means a gene that contributes to a cell being pluripotent.

“Pluripotent cell cultures” are said to be “substantially undifferentiated” when that display morphology that clearly distinguishes them from differentiated cells of embryo or adult origin. Pluripotent cells typically have high nuclear/cytoplasmic ratios, prominent nucleoli, and compact colony formation with poorly discernable cell junctions, and are easily recognized by those skilled in the art. It is recognized that colonies of undifferentiated cells can be surrounded by neighboring cells that are differentiated. Nevertheless, the substantially undifferentiated colony will persist when cultured under appropriate conditions, and undifferentiated cells constitute a prominent proportion of cells growing upon splitting of the cultured cells. Useful cell populations described in this disclosure contain any proportion of substantially undifferentiated pluripotent cells having these criteria. Substantially undifferentiated cell cultures may contain at least about 20%, 40%, 60%, or even 80% undifferentiated pluripotent cells (in percentage of total cells in the population).

“Regulatory protein” means any protein that regulates a biological process, including regulation in a positive and negative direction. The regulatory protein can have direct or indirect effects on the biological process, and can either exert affects directly or through participation in a complex.

“Reprogramming” means removing epigenetic marks in the nucleus, followed by establishment of a different set of epigenetic marks. During development of multicellular organisms, different cells and tissues acquire different programs of gene expression. These distinct gene expression patterns appear to be substantially regulated by epigenetic modifications such as DNA methylation, histone modifications and other chromatin binding proteins. Thus each cell type within a multicellular organism has a unique epigenetic signature that is conventionally thought to become “fixed” and immutable once the cells differentiate or exit the cell cycle. However, some cells undergo major epigenetic “reprogramming” during normal development or certain disease situations.

“Small molecule modulator” is meant to encompass compounds that are small molecule inhibitors or small molecule activators. A small molecule modulator may function as a small molecule inhibitor in some physiological contexts and as a small molecule activator in another physiological context. A small molecule modulator may function as a small molecule inhibitor with regard to one target, and as a small molecule activator with regard to another target. The same small molecule modulator may function as both a small molecule activator and as a small molecule inhibitor.

“Totipotent” means capable of developing into a complete embryo or organ.

Embodiments of the invention relate to methods comprising inducing expression of at least one gene that contributes to a cell being pluripotent or multipotent. In some embodiments, the methods induce expression of at least one gene that contributes to a cell being pluripotent or multipotent and producing reprogrammed cells that are capable of directed differentiation into at least one lineage.

Embodiments of the invention also relate to a method comprising modifying chromatin structure, and reprogramming a cell to be pluripotent or multipotent. In yet another embodiment, modifying chromatin structure comprises using a small molecule modulator to alter the activity of at least one regulatory protein involved in the regulation of transcription.

In yet another embodiment, modifying chromatin structure comprises using a small molecule inhibitor to inhibit the activity of at least one protein involved in transcriptional repression.

Embodiments of the invention also relate to a method comprising modifying a promoter region or upstream DNA sequence of a gene that contributes to a cell being pluripotent or multipotent. In still another embodiment, modifying the promoter structure or upstream DNA sequence comprises using a small molecule modulator to alter the activity, expression or activity and expression of at least one regulatory protein involved in transcription.

In another embodiment, the invention relates to a method comprising using a small molecule modulator to alter the activity, expression, or activity and expression of at least one regulatory protein involved in transcription, and inducing expression of at least one gene that contributes to a cell being pluripotent or multipotent. In yet another embodiment, the method comprises inhibiting the activity of at least one DNA methyltransferase and producing a reprogrammed cell.

In still another embodiment, the method comprises using a small molecule inhibitor to inhibit the activity of at least one DNA methyltransferase, demethylating at least one cytosine in a CpG dinucleotide, and inducing the expression of at least one gene that contributes to a cell being pluripotent or multipotent.

In yet another embodiment, the method comprises contacting a cell with a small molecule inhibitor; inhibiting the activity of at least one protein involved in transcriptional repression; and inducing the expression of at least one gene that contributes to a cell being pluripotent or multipotent. In yet another embodiment, the method further comprises producing a reprogrammed cell. The reprogrammed cell can be pluripotent or multipotent.

In still another embodiment, the invention relates to a method for reprogramming a cell comprising: exposing a cell to a small molecule modulator that induces expression of a pluripotent or multipotent gene; and selecting a cell, wherein differentiation potential has been restored to said cell. In yet another embodiment, the invention relates to a method for reprogramming a cell comprising: exposing a cell to a small molecule modulator that alters the activity, the expression, or the activity and expression of at least one regulatory protein, inducing the expression of a pluripotent or multipotent gene, and selecting a cell, wherein differentiation potential has been restored to said cell. The pluripotent or multipotent gene may be induced by any fold increase in expression including but not limited to 0.25-0.5, 0.5-1, 1.0-2.5, 2.5-5, 5-10, 10-15, 15-20, 20-40, 40-50, 50-100, 100-200, 200-500, and greater than 500. In another embodiment, the method comprises plating differentiated cells, exposing said differentiated cell to a small molecule modulator, culturing said cells, and identifying a cell that has been reprogrammed.

In another embodiment, altering the activity, expression, or expression and activity of a regulatory protein can lead to an increase in the activity of a regulatory protein, an increase in the expression of a regulatory protein, a decrease in the activity of a regulatory protein or a decrease in the expression of a regulatory protein. The activity or expression of a regulatory protein can be increased or decreased by any amount including but not limited to 1-5%, 5-10%, 10-20%, 20-30%, 30-40%, 40-50%, 50-60%, 60-70%, 70-80%, 80-90%, 90-95%, and 95-99%, 99-200%, 200-300%, 300-400%, 400-500% and greater than 500%.

In yet another embodiment, the method further comprises selecting a cell using an antibody directed to a protein or a fragment of a protein coded for by a pluripotent or multipotent gene or an antibody directed to a pluripotent or multipotent marker. Any type of antibody can be used including but not limited to a monoclonal, a polyclonal, a fragment of an antibody, a peptide mimetic, an antibody to the active region, and an antibody to the conserved region of a protein In still another embodiment, the method comprises selecting a cell and expanding or culturing said cell to a pluripotent cell culture.

In still another embodiment, the method further comprises selecting a cell using a reporter driven by a pluripotent or mulitpotent gene or a pluripotent or mulitpotent surface marker. Any type of reporter can be used including but not limited to a fluorescent protein, green fluorescent protein, cyan fluorescent protein (CFP), a yellow fluorescent protein (YFP), bacterial luciferase, jellyfish aequorin, enhanced green fluorescent protein, chloramphenicol acetyltransferase (CAT), dsRED, β-galactosidase, and alkaline phosphatase.

In still another embodiment, the method further comprises selecting a cell using resistance as a selectable marker including but not limited to resistance to an antibiotic, a fungicide, puromycin, hygromycin, dihydrofolate reductase, thymidine kinase, neomycin resistance (neo), G418 resistance, mycophenolic acid resistance (gpt), zeocin resistance protein and streptomycin.

In still another embodiment, the method further comprises comparing the chromatin structure of a pluripotent or multipotent gene of a cell that exists before exposure to a small molecule modulator to the chromatin structure of a pluripotent or multipotent gene obtained after treatment with a small molecule modulator. Any aspect of chromatin structure can be compared including but not limited to euchromatin, heterochromatin, histone acetylation, histone methylation, the presence and absence of histone or histone components, the location of histones, the arrangement of histones, and the presence or absence of regulatory proteins associated with chromatin. The chromatin structure of any region of a gene may be compared including but not limited to an enhancer element, an activator element, a promoter, the TATA box, regions upstream of the start site of transcription, regions downstream of the start site of transcription, exons and introns.

A small molecule inhibitor or “small molecular compound” refers to a compound useful in the methods, compositions, and kits of the invention having measurable or inhibiting activity. In some embodiments, the small molecule inhibitors have a relative molecular weight of not more than 1000 D, and in still other embodiments, of not more than 500 D. The small molecule inhibitor can be of organic or inorganic nature. In addition to small organic and inorganic compounds, peptides, antibodies, cyclic peptides and peptidomimetics are contemplated as being useful in the disclosed methods.

The small molecule modulator may be any of the compounds contained in a small molecule library or a modified compound derived from a compound contained in small molecule libraries. Several small molecule libraries are available from commercial sources including but not limited to BIOMOL INTERNATIONAL (now Enzo Life Sciences), and include but are not limited to Bioactive Lipid Library, Endocannabinoid Library, Fatty acid library, ICCB Known Bioactives Library, Ion Channel Ligand Library, Kinase Inhibitor Library, Kinase/Phosphatase Inhibitor Library, Neurotransmitter Library, Natural Products Library, Nuclear Receptor Library, Orphan Ligand Library, Protease Inhibitor Library, Phosphatase Inhibitor Library, and Rare Natural Products Library.

Small molecule inhibitors can be used to inhibit any protein involved in transcriptional repression including but not limited to histone deacetylases (HDAC), methyl binding domain proteins (MBD), methyl adenosyltransferases (MAT), DNA methyltransferases (DNMT), histone methyltransferase, and methyl cycle enzymes.

Preferably, such inhibition is specific, i.e., for a DNMT small molecule inhibitor, the DNMT inhibitor reduces the ability of a DNA methyltransferase to methylate a particular substrate or reduces the ability of a DNA methyltransferase to interact with another component required for methylation, at a concentration that is lower than the concentration of the inhibitor that is required to produce another, unrelated biological effect. Preferably, the concentration of the inhibitor required for DNA methyltransferase inhibitory activity is at least 2-fold lower, more preferably at least 5-fold lower, even more preferably at least 10-fold lower, and most preferably at least 20-fold lower than the concentration required to produce an unrelated biological effect.

Any number, any combination and any concentration of small molecule modulators can be used to alter the activity, expression, or activity and expression of a protein or more than one protein involved in transcriptional regulation including but not limited to 1, 2, 3, 4, 5, 6, 7, 8, 9, 10, 11-15, 16-20, 21-25, 25-50, 50-100, 100-250, and greater than 250. The small molecule modulator can be directed toward a specific protein or more than one protein, a specific class of proteins or more than one class of proteins, a specific family of proteins or more than one family of proteins or general transcriptional components.

A small molecule modulator may have an irreversible mechanism of action or a reversible mechanism of action. A small molecule modulator can have any binding affinity including but not limited to millimolar (mM), micromolar (EM), nanomolar (nM), picomolar (pM), and fentamolar (fM). A small molecule modulator can bind to a regulatory region or a catalytic region of the protein.

A representative list of proteins that may be inhibited by the methods of the invention is provided in Table I.

TABLE I
Representative list of Proteins involved in transcriptional repression

| | Methyl | | | | |
|---|---|---|---|---|---|
| | Binding | Meth. | | Histone | |
| Histone | Domain | Adenosyl- | DNA Methyl- | Methyl- | Methyl Cycle |
| Deacetylases | Proteins | transferases | transferases | transferase | Enzymes |
| Class 1 | MBD1 | MAT2A | DNMT1 | EHMT1 | MTHFR |
| (HDACs 1- | | | | | |
| 3, 8, 11 | | | | | |
| Class II | MBD2 | MAT1A | DNMT2 | HDM | CBS |
| (HDACs 4- | | | | G9A | |
| 7, 9, 10) | | | | | |
| Class III | MBD3 | MAT2B | DNMT3A | SUV39H1 | |
| (SIRTI 1-7) | | | | | |
| Class IV | MBD4 | | DNMT3B | SETDB1 | |
| (HDAC 11) | | | | | |
| | MeCP2 | | DNMT3L | | |

For example, a methyl binding domain protein, e.g., MeCP2, binds to methylated cytosines and recruits histone deacetylases that then deacetylate histone proteins, resulting in a condensed chromatin structure, which inhibits transcription. The methods of the invention can inhibit a protein involved in transcriptional repression and thereby induce transcription of pluripotency genes.

Therefore, based on the above representative description of a repression complex, a small molecule inhibitor can be used to inhibit the activity of MeCP2, thereby significantly reducing the recruitment of HDACs to chromatin structure. This can lead to the up-regulation of genes critical for a cell being pluripotent or multipotent and thereby increase differentiation capacity in somatic cells. Similarly, a small molecule inhibitor can be used to inhibit a DNMT, which would similarly lead to the up-regulation of genes that contribute to a cell being pluripotent or multipotent. Moreover, a small molecule inhibitor directed toward a DNA methyltransferase, and a small molecule inhibitor directed toward a methyl binding protein can be used simultaneously or sequentially to reduce the activity of at least one protein involved in repression complexes, which could lead to the induction of a pluripotent gene, and hence, the reprogramming of a cell. The above discussion is meant for illustrative purposes only and should not be construed to limit the scope of the invention.

A DNMT small molecule inhibitor may interact with and inhibit any DNA methyltransferase including but not limited to DNMT1, DNMT2, DNMT3A, and DNMT3B, and DNMT3L. DNMT1 is likely the most abundant DNA methyltransferase in mammalian cells, and considered to be involved in maintenance methyltransferase in mammals. DNMT1 predominantly methylates hemimethylated CpG di-nucleotides in the mammalian genome. The enzyme is 7-20 fold more active on hemimethylated DNA as compared with unmethylated substrate in vitro, but it is still more active at de novo methylation than other DNMTs. The enzyme is about 1620 amino acids long. The first 1100 amino acids constitute the regulatory domain of the enzyme, and the remaining residues constitute the catalytic domain. These are joined by Gly-Lys repeats. Both domains are required for the catalytic function of DNMT1.

DNMT2 has strong sequence similarities with 5-methylcytosine methyltransferases of both prokaryotes and eukaryotes. DNMT2 also has been shown to methylate position 38 in aspartic acid transfer RNA.

DNMT3 is a family of DNA methyltransferases that can methylate hemimethylated and unmethylated CpG dinucleotides at the same rate. The architecture of DNMT3 enzymes is similar to DNMT1 with regulatory region attached to a catalytic domain. DNMT3A and DNMT3B are responsible for the establishment of DNA methylation patterns during development. The DNMT3A and DNMT3B proteins are expressed at different stages of embryogenesis. DNMT3B appears to be expressed in totipotent embryonic cells, such as inner cell mass, epiblast and embryonic ectoderm cells, while DNMT3A appears to be ubiquitously expressed after E10.5

DNMT3L contains DNA methyltransferase motifs and is involved in establishing maternal genomic imprints. DNMT3L is also thought to play a role in transcriptional repression.

A DNMT small molecule inhibitor used in the methods, compositions, and kits of the invention may interact with a DNMT1, DNMT2, DNMT3A, DNMT3B or DNMT3L. A DNMT inhibitor may interact with one type of DNMT, all types of DNMTs or with multiple types of DNMTs including but not limited to including DNMT1 and DNMT2; DNMT1 and DNMT3A; DNMT1 and DNMT3B; DNMT1 and DNMT3L; DNMT2 and DNMT3A, DNMT2 and DNMT3B, DNMT2 and DNMT3L; DNMT3A and DNMT3B, DNMT3A and DNMT3L, DNMT3B and DNMT3L; DNMT1, DNMT2, DNMT3A; DNMT1, DNMT2, and DNMT3B; DNMT1, DNMT2, and DNMT3L; DNMT2, DNMT3A, and DNMT3B, DNMT2, DNMT3A and DNMT3L; DNMT2, DNMT3B, and DNMT3L and DNMT1, DNMT2, DNMT3A, and DNMT3B. A DNMT inhibitor of the invention may also interact with a DNMT that does not fall into one of the known types or is of yet unclassified.

In another embodiment, the DNMT inhibitor may act by binding to the regulatory domain or the catalytic domain of a DNMT. In another embodiment, the DNMT inhibitor may be a nucleoside analogue (incorporated into the DNA or RNA) or a non-nucleoside analogue. In another embodiment, the DNMT inhibitor may be an anti-sense oligonucleotide to a DNMT including but not limited to DNMT1, DNMT2, DNMT3A, DNMT3B, or DNMT3L. In still yet another embodiment, a DNMT inhibitor also may act by blocking protein-protein interactions.

In yet another embodiment, to inhibit Dnmt activity and cytosine methylation, cells may be grown in the following media:

(a) media treated with Dnmt1, 2, 3a, and/or 3b siRNA (Dharmacon, Inc.);

(b) media treated with RG108 (Analytical Systems Laboratory, LSU School of Veterinary Medicine);

(c) media treated with 5-AzadCyd (Sigma).

Table II provides a representative list of small molecule inhibitors that can inhibit a DNMT. A DNMT inhibitor used in the methods, compositions, and kits of the invention include derivatives and analogues of a DNMT inhibitor herein mentioned.

TABLE II
Representative Examples of Nucleoside Analogues and Non-
Nucleoside Analogues that are DNMT Inhibitors

| Nucleoside Analogues | Non-Nucleoside Analogues |
|---|---|
| 5-Azacytidine | Hydralazin |
| 5-aza-2-deoxycytidine | Hydralazine Hydrochloride |
| 5-Fluoro-2-doecytodine | Procainamide |
| 5,6-dihydro-5-azacytidine, | Procaine |
| 5-fluroouracil | |
| Zebularine | Procaine Hydrochloride |
| | Epigallocatechin-3-gallate |
| | (EFOG) |
| | Psammaplin A |
| | MG98 |
| | RG108 |

Table III is a representative list of small molecule modulators that can be used to induce, up-regulate or alter the expression of a gene involved in reprogramming. The small molecule modulator may target a component of the basal transcriptional machinery, a component of transcriptional activation, a component of a chromatin remodeling complex, a component of transcriptional repression, a component of DNA repair, a component of mismatch repair, and a component involved in maintaining the methylation state of a cell. Small molecule modulators include but are not limited to a histone deacetylase inhibitor (HDACi), a histone acetyltransferase inhibitor (HATi), a histone acetyltransferase activator, a lysine methyltransferase inhibitor (LMTi), a histone methyltransferase inhibitor (HMTi), a Trichostatin A inhibitor (TSAi), a histone demethylase inhibitor (HdeMi), a lysine demethylase inhibitor (LdeMi), a sirtuin inhibitor (SIRTi), and a sirtuin activator (SIRTa).

TABLE III
Representative list of small molecule modulators that can be used
to reprogram a cell

| Small Molecule Modulator | Function |
|---|---|
| HC Toxin | HDACi |
| Garcinol | HATi |
| BML-210 | HDACi |
| Chaetocin | LMTi/HMTi |
| ITSA1 | TSAi |
| Depudecin | HDACi |
| Tranylcypromine | HdeMi/LdeMi |
| EX-527 | SIRT1i |
| Resveratrol | SIRT1a |
| M-344 | HDACi |
| Nicotinamide | SIRTi |
| Fluoro-SAHA | HDACi |
| Piceatanol | SIRTa |
| BML-266 | SIRT2i |
| Sirtinol | SIRT2i |
| Valproxam | HDACi |
| AGK2 | SIRT2i |

Any small molecule modulator that functions as a histone acetyltransferase inhibitor can be used including but not limited to anacardic acid, garcinol, curcumin, isothiazolones, butyrolactone, and MC1626 (2-methyl-3-carbethoxyquinoline), polyisoprenylated Benzophenone, epigallocatechin-3-gallate (EGCG), and CPTH2 (cyclopentylidene-[4-(4′-chlorophenyl)thiazol-2-yl)hydrazone).

Any small molecule modulator that functions as a histone demethylase inhibitor can be used including but not limited to lysine specific demethylase, LSD1 (KIAA0601 or BHC110), flavin-dependent amine oxidase, and jumonji.

Any small molecule modulator that functions as a sirtuin activator can be used including but not limited to resveratrol, a polyphenol, a sirtuin activating compound, activators of SIRT1-SIRT7, and SRT-1720.

In one embodiment, the DNA methylation inhibitor is a cytidine analog or derivative. Examples of the cytidine analog or derivative include but art not limited to 5-azacytidine and 5-aza-2′-deoxycytidine (5-aza-CdR or decitabine).

5-aza-CdR is an antagonist of its related natural nucleoside, deoxycytidine. The only structural difference between these two compounds is the presence of a nitrogen at position 5 of the cytosine ring in 5-aza-CdR as compared to a carbon at this position for deoxycytidine. 5-aza-CdR functions as a mechanism-dependent suicide inhibitor of DNA methyltransferases. To be most effective, 5-aza-CdR is incorporated into DNA, which may require modification of the compound through metabolic pathways. DNA methyltransferases recognize 5-azacytosine as natural substrate and initiate the methylation reaction. However, the analogue prevents the resolution of a covalent reaction intermediate and the enzyme thus becomes trapped and degraded.

Zebularine, also known as 1-p-ribofuranosyl-1,2-dihydropyrimidin-2-one and 1-β-ribofuranosyl-2(1H)-pyrimidinone, has been attributed with cytidine deaminase inhibiting activity (see, e.g., Kim et al., J. Med. Chem. 29:1374-1380, 1986; McCormack et al., Biochem Pharmacol. 29:830-832, 1980).

Any number, any combination and any concentration of DNMT inhibitors can be used to inhibit a protein or more than one protein including but not limited to 1, 2, 3, 4, 5, 6, 7, 8, 9, 10, 11-15, 16-20, and 21-25.

Proteins in other complexes involved in chromatin remodeling also can be inhibited by methods of the invention including but not limited to the SWI/SNF complex, the NuRD complex, the Sin3 complex, and INO80. The hSWI/SNF complex is a multisubunit protein complex that is known to play a key role in regulation of chromatin accessibility. Any component of the hSWI/SNF complex can be inhibited by the methods of the invention including but not limited to SNF5/INI1, BRG1, BRM, BAF155, and BAF170. SWI/SNF was originally identified in yeast as required for activation of a variety of genes. The hSWI/SNF complexes have been shown to be essential for regulation of several developmentally specific gene expression programs.

Any component of the Sin3 complex can be inhibited by the methods of the invention including but not limited to HDAC1, HDAC2, RbAp46, RbAp48, Sin3A, SAP30, and SAP18.

Any component of the NuRD complex can be inhibited by the methods of the invention including but not limited to Mi2, p70, and p32.

Any component of the INO80 complex can be inhibited by the methods of the invention including but not limited to Tip49A, Tip49B, the SNF2 family helicase Ino80, actin related proteins ARP4, ARP5, and Arp8, YEATS domain family member Taf14, HMG-domain protein, Nhp10, and six additional proteins designated Iesl-6.

Any number of small molecule modulators can be used to induce the expression of a gene that contribute to a cell being pluripotent or multipotent including but not limited to 1-5, 6-10, 11-15, 16-20, 21-25, 26-30, 31-35, 36-40, 41-45, 46-50, and greater than 50 small molecule modulators.

The invention provides a reprogrammed cell that is obtained in the absence of eggs, embryos, embryonic stem cells, or somatic cell nuclear transfer (SCNT). A reprogrammed cell produced by the methods of the invention may be pluripotent or multipotent. A reprogrammed cell produced by the methods of the invention can have a variety of different properties including embryonic stem cell like properties. For example, a reprogrammed cell may be capable of proliferating for at least 10, 15, 20, 30, or more passages in an undifferentiated state. In other forms, a reprogrammed cell can proliferate for more than a year without differentiating. Reprogrammed cells can also maintain a normal karyotype while proliferating and/or differentiating. Some reprogrammed cells also can be cells capable of indefinite proliferation in vitro in an undifferentiated state. Some reprogrammed cells also can maintain a normal karyotype through prolonged culture. Some reprogrammed cells can maintain the potential to differentiate to derivatives of all three embryonic germ layers (endoderm, mesoderm, and ectoderm) even after prolonged culture. Some reprogrammed cells can form any cell type in the organism. Some reprogrammed cells can form embryoid bodies under certain conditions, such as growth on media that do not maintain undifferentiated growth. Some reprogrammed cells can form chimeras through fusion with a blastocyst, for example.

Reprogrammed cells can be defined by a variety of markers. For example, some reprogrammed cells express alkaline phosphatase. Some reprogrammed cells express SSEA-1, SSEA-3, SSEA-4, TRA-1-60, and/or TRA-1-81. Some reprogrammed cells express Oct 4, Sox2, and Nanog. It is understood that some reprogrammed cells will express these at the mRNA level, and still others will also express them at the protein level, on for example, the cell surface or within the cell.

A reprogrammed cell can have any combination of any reprogrammed cell property or category or categories and properties discussed herein. For example, a reprogrammed cell can express alkaline phosphatase, not express SSEA-1, proliferate for at least 20 passages, and be capable of differentiating into any cell type. Another reprogrammed cell, for example, can express SSEA-1 on the cell surface, and be capable of forming endoderm, mesoderm, and ectoderm tissue and be cultured for over a year without differentiation.

A reprogrammed cell can be alkaline phosphatase (AP) positive, SSEA-1 positive, and SSEA-4 negative. A reprogrammed cell also can be Nanog positive, Sox2 positive, and Oct-4 positive. A reprogrammed cell also can be Tcll positive, and Tbx3 positive. A reprogrammed cell can also be Cripto positive, Stellar positive and Dazl positive. A reprogrammed cell can express cell surface antigens that bind with antibodies having the binding specificity of monoclonal antibodies TRA-1-60 (ATCC HB-4783) and TRA-1-81 (ATCC HB-4784). Further, as disclosed herein, a reprogrammed cell can be maintained without a feeder layer for at least 10, 11, 12, 13, 14, 15, 16, 17, 18, 19, 20 passages or for over a year.

A reprogrammed cell may have the potential to differentiate into a wide variety of cell types of different lineages including fibroblasts, osteoblasts, chondrocytes, adipocytes, skeletal muscle, endothelium, stroma, smooth muscle, cardiac muscle, neural cells, hemiopoetic cells, pancreatic islet, or virtually any cell of the body. A reprogrammed cell may have the potential to differentiate into all cell lineages. A reprogrammed cell may have the potential to differentiate into any number of lineages including 1, 2, 3, 4, 5, 6-10, 11-20, 21-30, and greater than 30 lineages.

Any gene and associated family members of that gene that contribute to a cell being pluripotent or multipotent may be induced by the methods of the invention including but not limited to glycine N-methyltransferase (Gnmt), Octamer-4 (Oct4), Nanog, GABRB3, LEFTB, NR6A1, PODXL, PTEN, SRY (sex determining region Y)-box 2 (also known as Sox2), Myc, REX-1 (also known as Zfp-42), Integrin α-6, Rox-1, LIF-R, TDGF1 (CRIPTO), SALL4 (sal-like 4), Leukocyte cell derived chemotaxin 1 (LECTI), BUBI, FOXD3, NR5A2, TERT, LIFR, SFRP2, TFCP2L1, LIN28, XIST, and Krüppel-like factors (Klf) such as Klf4 and Klf5. Any number of genes that contribute to a cell being pluripotent or multipotent can be induced by the methods of the invention including but not limited to 1, 2, 3, 4, 5, 6, 7, 8, 9, 10, 11-20, 21-30, 31-40, 41-50, and greater than 50 genes.

Further, Ramalho-Santos et al. (Science 298, 597 (2002), Ivanova et al. (Science 298, 601 (2002) and Fortunel et al. (Science 302, 393b (2003)) (all incorporated by reference in their entirety) each compared three types of stem cells and identified a list of commonly expressed “stemness” genes, proposed to be important for conferring the functional characteristics of stem cells. Any of the genes identified in the above-mentioned studies may be induced by the methods of the invention. Table III provides a list of genes thought to be involved in conferring the functional characteristics of stem cells. In addition to the genes listed in Table IV, 93 expressed sequence tags (EST) clusters with little or no homology to known genes were also identified by Ramalho-Santos et al. and Ivanova et al., and are included within the methods of the invention.

TABLE IV
Genes implicated in conferring stem cell characteristics

| symbol | Gene | Function |
|---|---|---|
| F2r | Thrombin receptor | G-protein coupled receptor, coagulation |
| | | cascade, required for vascular development |
| Ghr | Growth hormone | Growth hormone receptor/binding protein, |
| | receptor | activates Jak2 |
| Itga6 | Integrin alpha 6 | cell adhesion, cell-surface mediated |
| | | signalling, can combine with Integrin b1 |
| Itgb1 | Integrin beta 1 | cell adhesion, cell-surface mediated |
| | (fibronectin Receptor) | signalling, can combine with Integrin a6 |
| Adam 9 | A disintegrin and | cell adhesion, extracellular proteolysis, |
| | metalloproteinase | possible fusogenic function |
| | domain 9 (meltrin | |
| | gamma) | |
| Bys | Bystin-like (Bystin) | cell adhesion, may be important for |
| | | embryo implantation (placenta) |
| Ryk | Receptor-like tyrosine | unconventional receptor tyrosine kinase |
| | kinase | |
| Pkd2 | Polycystic kidney | calcium channel |
| | disease 2 | |
| Kcnab3 | Potassium voltage gated | Regulatory subunit of potassium channel |
| | channel, shaker related | |
| | subfamily, beta member 3 | |
| Gnb1 | Guanine nucleotide | G-protein coupled receptor signaling |
| | binding protein beta 1 | |
| Gab1 | Growth factor receptor | integration of multiple signaling pathways |
| | bound protein 2 (Grb2)- | |
| | associated protein 1 | |
| Kras2 | Kirsten rat sarcoma | binds GTP and transmits signals from |
| | oncogene 2 | growth factor receptors |
| | ESTs highly similar to | suppressor of RAS function |
| | Ras p21 protein | |
| | activator (Gap) | |
| Cttn | Cortactin | regulates actin cytoskeleton, overexpressed |
| | | in tumors |
| Cops4 | COP9 (constitutive | Cop9 signalosome, integration of multiple |
| | photomorphogenic), | signaling pathways, regulation of protein |
| | subunit 4 | degradation |
| Cops7a | COP9 (constitutive | Cop9 signalosome, integration of multiple |
| | photomorphogenic), | signaling pathways, regulation of protein |
| | subunit 7a | degradation |
| Madh1 | Mad homolog 1 | TGFb pathway signal transducer |
| | (Smad1) | |
| Madh2 | Mad homolog 2 | TGFb pathway signal transducer |
| | (Smad2) | |
| Tbrg1 | TGFb regulated 1 | induced by TGFb |
| Stam | signal transducing | Associates with Jak tyrosine kinase |
| | adaptor molecule (SH3 | |
| | domain and ITAM | |
| | motif) 1 | |
| Statip1 | STAT interacting | scaffold for Jak/Stat3 binding |
| | protein 1 | |
| Cish2 | Cytokine inducible SH2- | STAT induced STAT inhibitor-2, interacts |
| | containing protein 2 | with Igf1R |
| | (Ssi2) | |
| | ESTs moderately similar | possible tyrosine kinase |
| | to Jak3 | |
| | ESTs highly similar to | regulatory subunit of protein phosphatase |
| | PPP2R1B | 2, putative tumor suppressor |
| Rock2 | Rho-associated coiled- | serine/theonine kinase, target of Rho |
| | coil forming kinase 2 | |
| Yes | Yamaguchi sarcoma | intracellular tyrosine kinase, proto- |
| | viral oncogene homolog | oncogene, Src family |
| Yap | Yes-associated protein 1 | bind Yes, transcriptional co-activator |
| Ptpn2 | Protein tyrosine non- | dephosphorylates proteins |
| | receptor phosphatase 2 | |
| Ppplr2 | Protein phosphatase 1, | Inhibitory subunit of protein phosphatase 1 |
| | regulatory (inhibitor) 2 | |
| Ywhab | Tyrosine/tryptophan | Binds phosphoserine-proteins, PKC |
| | monooxgenase | pathway |
| | activation protein beta | |
| | (14-3-3beta) | |
| Ywhah | Tyrosine/tryptophan | Binds phosphoserine-proteins, PKC |
| | monooxgenase | pathway |
| | activation protein eta | |
| | (14-3-3eta) | |
| Axo | Axotrophin | contains a PHD domain, an adenylaye |
| | | cyclase domain and a consensus region for |
| | | G-protein interaction, required for neuronal |
| | | maintenance |
| Trip6 | Thyroid hormone | interacts with THR in the presence of TH, |
| | receptor interactor 6 | putative co-activator for Rel transcription |
| | | factor |
| Gfer | Growth factor, erv1 (S. cerevisiae)- | sulphydryl oxidase, promotes liver |
| | like | regeneration, stimulates EGFR and MAPK |
| | (augmenter of liver | pathways |
| | regeneration) | |
| Upp | Uridine phosphorylase | Interconverts uridine and uracil, highly |
| | | expressed in transformed cells, may |
| | | produce 2-deoxy-D-ribose, a potent |
| | | angiogenic factor |
| Mdfi | MyoD family inhibitor | inhibitor of bHLH and beta-catenin/TCF |
| | | transcription factors |
| Tead2 | TEA domain 2 | transcriptional factor |
| Yap | Yes-associated 65 kD | Binds Yes, transcriptional co-activator |
| Fhl1 | Four and a half LIM | may interact with RBP-J/Su(H) |
| Zfx | Zinc Finger X-linked | zinc finger, putative transcription factor |
| Zfp54 | Zinc finger 54 | zinc finger, putative transcription factor |
| | Zinc finger protein | zinc finger, putative transcription factor |
| D17Ertd197e | D17Ertd197e | zinc finger, putative transcription factor |
| | ESTs, high similarity to | zinc finger, putative transcription factor |
| | Zfp | |
| | ESTs, high similarity to | zinc finger, putative transcription factor |
| | Zfp | |
| | ESTs, high similarity to | zinc finger, putative transcription factor |
| | Zfp | |
| Rnf4 | RING finger 4 | steroid-mediated transcription |
| Chd1 | Chromodomain helicase | modification of chromatin structure, |
| | DNA binding protein 1 | SNF2/SW12 family |
| Etl1 | enhancer trap locus 1 | modification of chromatin structure, |
| | | SNF2/SW12 family |
| Rmp | Rpb5-mediating protein | Binds RNA, PolII, inhibits transcription |
| Ercc5 | Excision repair 5 | Endonuclease, repair of UV-induced |
| | | damage |
| Xrcc5 | X-ray repair 5 (Ku80) | helicase, involved in V(D)J recombination |
| Msh2 | MutS homolog 2 | mismatch repair, mutated in colon cancer |
| Rad23b | Rad23b homolog | excision repair |
| Ccnd1 | Cyclin D1 | G1/S transition, regulates CDk2 and 4, |
| | | overexpressed in breast cancer, implicated |
| | | in other cancers |
| Cdkn1a | Cdk inhibitor 1a P21 | inhibits G1/S transition, Cdk2 inhibitor, |
| | | required for HSC maintenance |
| Cdkap1 | Cdk2 associated protein | binds DNA primase, possible regulator of |
| | | DNA replication (S phase) |
| Cpr2 | Cell cycle progression 2 | overcomes G1 arrest in S. cerevisiae |
| Gas2 | Growth arrest specific 2 | highly expressed in growth arrested cells, |
| | | part of actin cytoskeleton |
| CenpC | Centromere protein C | present in active centromeres |
| Wig1 | Wild-type p53 induced 1 | p53 target, inhibits tumor cell growth |
| Tmk | Thymidylate kinase | dTTP synthesis pathway, essential for S |
| | | phase progression |
| Umps | Uridine monophosphate | Pyrimidine biosynthesis |
| | synthetase | |
| Sfrs3 | Splicing factor RS rich 3 | implicated in tissue-specific differential |
| | | splicing, cell cycle regulated |
| | ESTs highly similar to | Cell cycle-regulated nuclear export protein |
| | exportin 1 | |
| | ESTs highly similar to | trifunctional protein of pyrimidine |
| | CAD | biosynthesis, activated (phosphorylated) by |
| | | MAPK |
| | ESTs similar to | Map kinase cascade |
| | Mapkkkk3 | |
| Gas2 | Growth arrest specific 2 | highly expressed in growth arrested cells, |
| | | part of actin cytoskeleton, target of |
| | | caspase-3, stabilizes p53 |
| Wig1 | Wild-type p53 induced 1 | p53 target, inhibits tumor cell growth |
| Pdcd2 | Programmed cell death 2 | Unknown |
| Sfrs3 | Splicing factor RS rich 3 | implicated in tissue-specific differential |
| | | splicing, cell cycle regulated |
| | ESTs highly similar to | putative splicing factor |
| | Sfrs6 | |
| | ESTs highly similar to | putative splicing factor |
| | pre-mRNA splicing | |
| | factor Prp6 | |
| Snrp1c | Small nuclear | U1 snRNPs, component of the spliceosome |
| | ribonucleoprotein | |
| | polypeptide C | |
| Phax | Phosphorylated adaptor | mediates U snRNA nuclear export |
| | for RNA export | |
| NOL5 | Nucleolar protein 5 (SIK | pre-rRNA processing |
| | similar) | |
| | ESTs highly similar to | pre-rRNA processing |
| | Nop56 | |
| Rnac | RNA cyclase | Unknown |
| | ESTs highly similar to | DEAD-box protein, putative RNA helicase |
| | Ddx1 | |
| Eif4ebp1 | Eukaryotic translation | translational repressor, regulated |
| | initiation factor 4E | (phosphorylated) by several signaling |
| | binding protein 1 | pathways |
| Eif4g2 | Eukaryotic translation | translational repressor, required for |
| | initiation factor 4, | gastrulation and ESC differentiation |
| | gamma 2 | |
| | ESTs highly similar to | Translation initiation factor |
| | Eif3s1 | |
| Mrps31 | Mitochondrial ribosomal | component of the ribosome, mitochondria |
| | protein S31 | |
| Mrpl17 | Mitochondrial ribosomal | component of the ribosome, mitochondria |
| | protein L17 | |
| Mrpl34 | Mitochondrial ribosomal | component of the ribosome, mitochondria |
| | protein L34 | |
| Hspa11 | Heat shock 70 kD | Chaperone, testis-specific |
| | protein-like 1 (Hsc70t) | |
| Hspa4 | Heat shock 70 kDa | Chaperone |
| | protein 4 (Hsp110) | |
| Dnajb6 | DnaJ (Hsp40) homolog, | co-chaperone |
| | subfamily B, member 6 | |
| | (Mammalian relative of | |
| | Dnaj) | |
| Hrsp12 | Heat responsive | possible chaperone |
| Tcp1-rs1 | T-complex protein 1 | possible chaperone |
| | related sequence 1 | |
| Ppic | Peptidylprolyl isomerase | Isomerization of peptidyl-prolyl bonds |
| | C (cyclophilin C) | |
| Fkbp9 | FK506-binding protein 9 | possible peptidyl-prolyl isomerase |
| | (63 kD) | |
| | ESTs moderately similar | possible peptidyl-prolyl isomerase |
| | to Fkbp13 | |
| Ube2d2 | Ubiquitin-conjugating | E2, Ubiquitination of proteins |
| | enzyme E2D2 | |
| Arih1 | Ariadne homolog | likely E3, Ubiquitin ligase |
| Fbxo8 | F-box only 8 | putative SCF Ubiquitin ligase subunit |
| | ESTs moderately similar | possible E2, Ubiquitination of proteins |
| | to Ubc13 (bendless) | |
| Usp9x | Ubiquitin protease 9, X | removes ubiquitin from proteins |
| | chromosome | |
| Uchrp | Ubiquitin c-terminal | likely removes ubiquitin from proteins |
| | hydrolase related | |
| | polypeptide | |
| Axo | Axotrophin | contains RING-CH domain similar to E3s, |
| | | Ubiquitin ligases |
| Tpp2 | Tripeptidyl peptidase II | serine expopeptidase, associated with the |
| | | proteasome |
| Cops4 | COP9 (constitutive | Cop9 signalosome, integration of multiple |
| | photomorphogenic) | signaling pathways, regulation of protein |
| | subunit 4 | degredation |
| Cops 7a | COP9 (constitutive | Cop9 signalosome, integration of multiple |
| | photomorphogenic), | signaling pathways, regulation of protein |
| | subunit 7a | degradation |
| | ESTs highly similar to | Regulatory subunit of the proteasome |
| | proteasome 26S subunit, | |
| | non-ATPase, 12 (p55) | |
| Nyren18 | NY-REN-18 antigen | interferon-9 induced, downregulator of |
| | (NUB1) | Nedd8, a ubiquitin-like protein |
| Rab18 | Rab18, member RAS | small GTPase, may regulate vesicle |
| | oncogene family | transport |
| Rabggtb | RAB geranlygeranyl | regulates membrane association of Rab |
| | transferase, b subunit | proteins |
| Stxbp3 | Syntaxin binding protein 3 | vesicle/membrane fusion |
| Sec23a | Sec23a (S. cerevisiae) | ER to Golgi transport |
| | ESTs moderately similar | ER to Golgi transport |
| | to Coatomer delta | |
| Abcb1 | Multi-drug resistance 1 | exclusion of toxic chemicals |
| | (Mdr1) | |
| Gsta4 | Glutathione S- | response to oxidative stress |
| | transferase 4 | |
| Gslm | Glutamate-cycteine | glutathione biosynthesis |
| | ligase modifier subunit | |
| Txnrd1 | Thioredoxin reductase | delivers reducing equivalents to |
| | | Thioredoxin |
| Txn1 | Thioredoxin-like 32 kD | redox balance, reduces dissulphide bridges |
| | | in proteins |
| Laptm4a | Lysosomal-associated | import of small molecules into lysosome |
| | protein transmembrane | |
| | 4A (MTP) | |
| Rcn | Reticulocalbin | ER protein, Ca+2 binding, overexpressed |
| | | in tumor cell lines |
| Supl15h | Suppressor of Lec15 | ER synthesis of dolichol phosphate- |
| | homolog | mannose, precursor to GPI anchors and N- |
| | | glycosylation |
| Pla2g6 | Phospholipase A2, | Hydrolysis of phospholipids |
| | group VI | |
| Acadm | Acetyl-Coenzyme A | fatty acid beta-oxidation |
| | dehydrogenase, medium | |
| | chain | |
| Suclg2 | Succinate-Coenzyme A | regulatory subunit, Krebs cycle |
| | ligase, GDP-forming, | |
| | beta subunit | |
| Pex7 | Peroxisome biogenesis | Peroxisomal protein import receptor |
| | factor 7 | |
| Gcat | Glycine C- | conversion of threonine to glycine |
| | acetyltransferase (KBL) | |
| Tjp1 | Tight junction protein 1 | component of tight junctions, interacts with |
| | | cadherins in cells lacking tight junctions |

Embodiments of the invention also include methods for treating a variety of diseases using a reprogrammed cell produced according to the novel methods disclosed elsewhere herein. The skilled artisan would appreciate, based upon the disclosure provided herein, the value and potential of regenerative medicine in treating a wide plethora of diseases including, but not limited to, heart disease, diabetes, skin diseases and skin grafts, spinal cord injuries, Parkinson's disease, multiple sclerosis, Alzheimer's disease, and the like. The present invention encompasses methods for administering reprogrammed cells to an animal, including humans, in order to treat diseases where the introduction of new, undamaged cells will provide some form of therapeutic relief.

The skilled artisan will readily understand that reprogrammed cells can be administered to an animal as a re-differentiated cell, for example, a neuron, and will be useful in replacing diseased or damaged neurons in the animal. Additionally, a reprogrammed cell can be administered to the animal and upon receiving signals and cues from the surrounding milieu, can re-differentiate into a desired cell type dictated by the neighboring cellular milieu. Alternatively, the cell can be re-differentiated in vitro and the differentiated cell can be administered to a mammal in need there of.

The reprogrammed cells can be prepared for grafting to ensure long term survival in the in vivo environment. For example, cells can be propagated in a suitable culture medium, such as progenitor medium, for growth and maintenance of the cells and allowed to grow to confluence. The cells are loosened from the culture substrate using, for example, a buffered solution such as phosphate buffered saline (PBS) containing 0.05% trypsin supplemented with 1 mg/ml of glucose; 0.1 mg/ml of MgCl.sub.2, 0.1 mg/ml CaCl.sub.2 (complete PBS) plus 5% serum to inactivate trypsin. The cells can be washed with PBS using centrifugation and are then resuspended in the complete PBS without trypsin and at a selected density for injection.

Formulations of a pharmaceutical composition suitable for peritoneal administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for peritoneal administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The invention also encompasses grafting reprogrammed cells in combination with other therapeutic procedures to treat disease or trauma in the body, including the CNS, PNS, skin, liver, kidney, heart, pancreas, and the like. Thus, reprogrammed cells of the invention may be co-grafted with other cells, both genetically modified and non-genetically modified cells which exert beneficial effects on the patient, such as chromaffin cells from the adrenal gland, fetal brain tissue cells and placental cells. Therefore the methods disclosed herein can be combined with other therapeutic procedures as would be understood by one skilled in the art once armed with the teachings provided herein.

The reprogrammed cells of this invention can be transplanted “naked” into patients using techniques known in the art such as those described in U.S. Pat. Nos. 5,082,670 and 5,618,531, each incorporated herein by reference, or into any other suitable site in the body.

The reprogrammed cells can be transplanted as a mixture/solution comprising of single cells or a solution comprising a suspension of a cell aggregate. Such aggregate can be approximately 10-500 micrometers in diameter, and, more preferably, about 40-50 micrometers in diameter. A reprogrammed cell aggregate can comprise about 5-100, more preferably, about 5-20, cells per sphere. The density of transplanted cells can range from about 10,000 to 1,000,000 cells per microliter, more preferably, from about 25,000 to 500,000 cells per microliter.

Transplantation of the reprogrammed cell of the invention can be accomplished using techniques well known in the art as well those developed in the future. The invention comprises a method for transplanting, grafting, infusing, or otherwise introducing reprogrammed cells into an animal, preferably, a human.

The reprogrammed cells also may be encapsulated and used to deliver biologically active molecules, according to known encapsulation technologies, including microencapsulation (see, e.g., U.S. Pat. Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 10⁵ to 10⁷ cells. Several macroencapsulation devices may be implanted in the patient. Methods for the macroencapsulation and implantation of cells are well known in the art and are described in, for example, U.S. Pat. No. 6,498,018.

Reprogrammed cells of the invention also can be used to express a foreign protein or molecule for a therapeutic purpose or for a method of tracking their integration and differentiation in a patient's tissue. Thus, the invention encompasses expression vectors and methods for the introduction of exogenous DNA into reprogrammed cells with concomitant expression of the exogenous DNA in the reprogrammed cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Embodiments of the invention also relate to a method for identifying regulators of the epigenome comprising contacting a cell with a small molecule library, measuring a change to the genome; and identifying the regulator of the genome. The method further comprises identifying the small molecule modulator. In still another embodiment, measuring a change to the genome includes but is not limited to acetylation, deacetylation, methylation, demethylation, phosphorylation, ubiquitination, sumoylation, ADP-ribosylation, and deimination.

Embodiments of the invention also relate to a composition comprising a cell that has been produced by the methods of the invention. In another embodiment, the invention relates to a composition comprising cell that has been reprogrammed by using a small molecule inhibitor to inhibit the activity of at least one protein involved in transcriptional repression. In yet another embodiment, the invention relates to a composition comprising a cell that has been reprogrammed by inducing the expression of a gene that contributes to a cell being pluripotent or multipotent.

Embodiments of the invention also relate to a reprogrammed cell that has been produced by contacting a cell with at least one small molecule modulator. In yet another embodiment, the invention relates to a reprogrammed cell that has been produced by contacting a cell with a small molecule inhibitor that inhibits at least one DNMT, including but not limited to RG108, 5-aza-2-deoxycytidine, and Epigallocatechin-3-gallate.

Embodiments of the invention also relate to kits for preparing the methods and compositions of the invention. The kit can be used for, among other things, producing a reprogrammed cell and generating ES-like and stem cell-like cells, inducing the expression of a gene that contributes to a cell being pluripotent or multipotent, and inhibiting the activity of at least one protein involved in transcriptional repression. The kit may comprise at least one small molecule inhibitor. The kit may comprise multiple small molecule inhibitors. The small molecule inhibitors can be provided in a single container or in multiple containers.

The kit may also comprise reagents necessary to determine if the cell has been reprogrammed including but not limited to reagents to test for the induction of a gene that contributes to a cell being pluripotent or multipotent, reagents to test for inhibition of a DNMT, regents to test for demethylating of CpG dinucleotides, and reagents to test for remodeling of the chromatin structure.

The kit may also comprise regents that can be used to differentiate the reprogrammed cell into a particular lineage or multiple lineages including but not limited to a neuron, an osteoblast, a muscle cell, an epithelial cell, and hepatic cell.

The kit may also contain an instructional material, which describes the use of the components provide in the kit. As used herein, an “instructional material” includes a publication, a recording, a diagram, or any other medium of expression that can be used to communicate the usefulness of the methods of the invention in the kit for, among other things, effecting the reprogramming of a differentiated cell. Optionally, or alternately, the instructional material may describe one or more methods of re- and/or trans-differentiating the cells of the invention. The instructional material of the kit of the invention may, for example, be affixed to a container that contains a small molecule inhibitor. Alternatively, the instructional material may be shipped separately from the container with the intention that the instructional material and a small molecule inhibitor, or component thereof, be used cooperatively by the recipient.

The invention is now described with reference to the following Examples. These Examples are provided for the purpose of illustration only and the invention should in no way be construed as being limited to these Examples, but rather should be construed to encompass any and all variations that become evident as a result of the teaching provided herein. All references including but not limited to U.S. patents, allowed U.S. patent applications, or published U.S. patent applications are incorporated within this specification by reference in their entirety.

### EXAMPLES

The following examples are illustrative only and are not intended to limit the scope of the invention as defined by the claims.

### Example 1

The ability of a small molecule modulator to induce or up-regulate pluripotency genes in human somatic cells was tested. In this example, the small molecule modulator was the small molecule inhibitor, RG108, which inhibits the activity of at least one DNMT. However, one of ordinary skill in the art will understand that any small molecule modulator that induces the expression of a pluripotent or mulitpotent gene could be used.

Methods

Cell culture. Primary human lung fibroblasts were purchased from Cell Applications (San Diego, Calif.), and were maintained at 37° C. in 95% humidity and 5% CO₂ in Dulbecco's modified eagle medium (DMEM, Hyclone) containing 10% fetal bovine serum (FBS, Hyclone) and 0.5% penicillin and streptomycin. Cells were grown in the presence of 500 μM RG108 for five days or left untreated.

Quantitative RT-PCR. Expression of Oct-4 and Nanog were determined by real-time RT-PCR for each culture condition (500 μM RG108 or untreated). Briefly, total RNA was prepared from cultures using Trizol Reagent (Life Technologies, Gaithersburg, Md.) and RNeasy Mini kit (Qiagen; Valencia, Calif.) with DNase I digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of Oct-4 and Nanog was normalized to glyceraldehyde 3-phosphate-dehydrogenase (GAPD).

Results

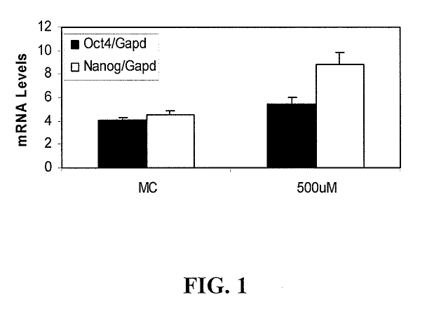

As shown in FIG. 1, treatment of primary human lung fibroblasts with 500 μM RG108 for 5 days resulted in significant (p<0.03) up regulation of Nanog gene expression. In addition, a trend for increased Oct4 gene expression (p<0.07) also was observed. Up-regulation of the pluripotent genes Oct-4 and Nanog also was observed by culturing cells in the presence of the DNMT inhibitor epigallocatechin-3-gallate (p<0.08; data not shown).

These results suggest that small molecule inhibitors can be used to regulate the epigenome, for example, DNA methylation. Small molecule inhibitors can be used to inhibit the activity of proteins involved in transcriptional repression. In addition, small molecule inhibitors can induce the expression of pluripotency genes and restore differentiation potential in somatic cells.

### Example 2

A variety of small molecule modulators were used to induce or up-regulate pluripotency genes in several cell types. In this example, Oct-4 was the primary gene examined; however, one of ordinary skill in the art will understand that small molecule modulators can be used to induce or up-regulate the expression of any gene involved in reprogramming.

Methods

Cell culture. Primary human dermal fibroblasts, adult and neonatal, were purchased from Cell Applications (San Diego, Calif.). Human lung fibroblasts, HSM cells and BJ fibroblasts were purchased from American Type Culture Collection (ATCC, Manassas, Va.).

Cells were maintained at 37° C. in 95% humidity and 5% CO₂ in Dulbecco's modified eagle medium (DMEM, Hyclone) containing 10% fetal bovine serum (FBS, Hyclone) and 0.5% penicillin and streptomycin or in Fibroblast Growth Medium (Cell Applications, San Diego, Calif.). Cells were grown in the presence or absence of a small molecule modulator. Culture time in the presence of the small molecule modulator varied for each small molecule modulator (see Table V).

Quantitative RT-PCR. Expression of the gene of interest, for example Oct-4, Nanog, or Sox-2, was determined by real-time RT-PCR for each culture condition. Briefly, total RNA was prepared from cultures using Trizol Reagent (Life Technologies, Gaithersburg, Md.) and RNeasy Mini kit (Qiagen; Valencia, Calif.) with DNase I digestion according to manufacturer's protocol. Total RNA (1 μg) from each sample was subjected to oligo(dT)-primed reverse transcription (Invitrogen; Carlsbad, Calif.). Real-time PCR reactions will be performed with PCR master mix on a 7300 real-time PCR system (Applied Biosystems; Foster City, Calif.). For each sample, 1 μl of diluted cDNA (1:10) will be added as template in PCR reactions. The expression level of the gene of interest was normalized to glyceraldehyde 3-phosphate-dehydrogenase (GAPD).

Results

Table V lists small molecule modulators that were tested and shown to induce or up-regulate the expression of Oct-4. The small molecule inhibitors, VPA and RG108, have also been shown to induce Nanog (see FIG. 1, and Table V). The data presented in Table V demonstrates that a large number of small molecule modulators can be used, at various concentrations, to induce or up-regulate the expression of pluripotency genes, such as Oct-4. As shown in Table V, small molecule modulators, at various concentrations and at various times of incubation, can induce Oct-4 expression in adult human dermal fibroblasts, neonatal human dermal fibroblasts, human lung fibroblasts, and BJ fibroblasts (human foreskin).

TABLE V
Small Molecule Modulators that increase expression of
pluripotent genes

| Small Molecule | | | | | Cell | Oct4 | | Nanog | |
|---|---|---|---|---|---|---|---|---|---|
| Modulator | Target | Dose | | Time (d) | type | FC | p < | FC | p < |
| VPA | Class I HDACi | 5 | mM | 5 | HDFa | 2.5 | 0.01 | | |
| VPA | Class I HDACi | 5 | mM | 3 | HLFa | 2.7 | 0.01 | | |
| VPA | Class I HDACi | 1 | mM | 3 | HDFn | ~4 | | ~2 | |
| VPA | Class I HDACi | 1 | mM | 3 | HSMa | <2 | | | |
| EGCG | DNMTi | 5 | ug/ml | 5 | HLFa | | | | 0.08 |
| RG108 | DNMTi | 500 | uM | 5 | HLFa | <2 | 0.07 | 2 | 0.03 |
| RG108 | DNMTi | 0.25, 0.5, 1 | mM | 3 | HDFn | 2-2.5 | | | |
| RG108 | DNMTi | 500 | uM | 3 | HDFa | <2 | | | |
| Hydralazine HCL | DNMTi | 500 | uM | 2 | HDFa | <2 | 0.05 | | |
| Hydralazine HCL | DNMTi | 50 | uM | 4 | HDFa | <2 | 0.07 | | |
| ALA | HDACi | 50 | uM | 4 | HDFa | ~3.5 | 0.0001 | | |
| Biotin | HDACi | 500 | uM | 4 | HDFa | <2 | 0.07 | | |
| Nicotinamide | SIRTi | 500 | uM | 4 | HDFa | <2 | 0.02 | | |
| Nicotinamide | SIRTi | .28 | mM | 4 | HDFa | <3 | 0.03 | | |
| Nicotinamide | SIRTi | .028 | mM | 4 | HDFa | 2.3 | 0.01 | | |
| Procaine HCL | DNMTi | 50 | uM | 2 | HDFa | <2 | 0.09 | | |
| Procaine HCL | DNMTi | 500 | uM | 8 | BJF | 1.6 | 0.02 | | |
| Procaine HCL | DNMTi | 1 | mM | 8 | BJF | 1.8 | 0.05 | | |
| Na | HDACi | 2.5 | mM | 4 | HDFa | 2.5 | 0.06 | | |
| Phenylbutyrate | | | | | | | | | |
| Tranylcypromine | LdeMi | 2.5 | mM | 4 | HDFa | 2.7 | | | |
| SIRT1 Activator 3 | SIRT1a | 500 | uM | 4 | HDFa | 2 | 0.01 | | |
| CAY 10433 | HDACi | 50 | uM | 4 | HDFa | ~.3 | 0.05 | | |
| Depudecin | HDACi | 50 | uM | 4 | HDFa | 1.6 | 0.01 | | |
| EX-527 | SIRTi | 50 | uM | 4 | HDFa | 2.1 | 0.01 | | |
| Splitomycin | HDACi | .5 | mM | 4 | HDFa | 2 | 0.02 | | |
| ITSA | TSAi | .05 | mM | 4 | HDFa | 1.3 | 0.03 | | |
| Valproxam | HDACi | .05 | mM | 4 | HDFa | 1.2 | 0.03 | | |
| Valproxam | HDACi | .5 | mM | 4 | HDFa | 1.5 | 0.02 | | |
| Resveratrol | SIRT1a | .05 | mM | 4 | BJF | 1.3 | 0.08 | | |
| Resveratrol | SIRT1a | .5 | mM | 4 | BJF | 1.8 | 0.01 | | |
| 2-PCPA | HDMi/LSD1i | 100 | uM | 8 | BJF | 1.4 | 0.06 | | |
| 2-PCPA | HDMi/LSD1i | 500 | uM | 8 | BJF | 1.8 | 0.07 | | |
| 2-PCPA | HDMi/LSD1i | 1 | mM | 8 | BJF | 1.7 | 0.001 | | |
| Tranylcypromine | HDMi/LSD1 | 2.5 | mM | 4 | HDFa | <3 | | | |
| EGCG | DNMT1 | 1 | uM | 4 | BJF | <2 | 0.09 | | |
| Ro-31-8220 | SIRTi | 2.5 | mM | 4 | BJF | | | | |

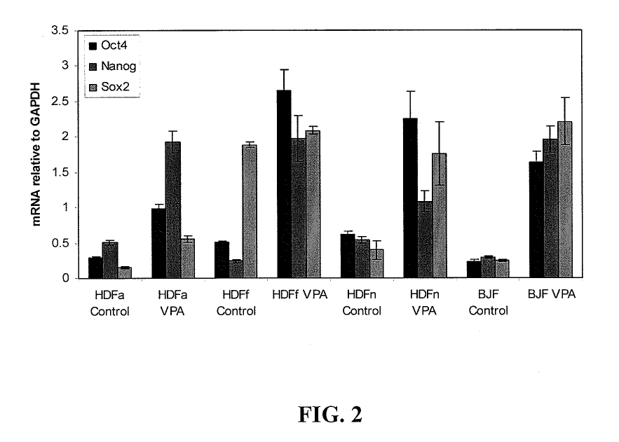

Valproic acid (VPA) (5 mM) induced the expression of Oct-4, Nanog, and Sox-2 in adult human dermal fibroblasts, neonatal human dermal fibroblasts, fetal human dermal fibroblasts, and BJ fibroblasts (see FIG. 2). Cells were treated with VPA for 4-6 days. The increase in expression varied for each gene and in each cell type; however, the data clearly show an up-regulation of pluripotency genes in the presence of a small molecule inhibitor (VPA). The house keeping gene, GAPDH, was used to normalize the amount of mRNA.

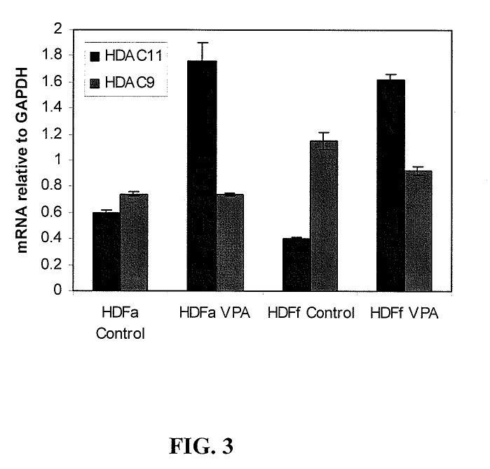

As shown in FIG. 3, VPA also increased the expression of HDAC 11 in adult and fetal human dermal fibroblasts. With regard to HDAC 9, there was no statistical difference between the VPA treated and untreated cells. The lack of a measurable effect may be due to experimental limitation imposed by the scientific equipment.

Table VI presents the statistical analysis of the pluripotency genes Oct-4, Nanog, Sox-2, and HDAC 11 in the presence of VPA. Information on four cell types is presented: adult human dermal fibroblasts, fetal human dermal fibroblasts, neonatal human dermal fibroblasts, and BJ fibroblasts. In each cell type, the change in the expression of Oct-4, Nanog, and Sox-2 was statistically significant. The expression of multiple genes, which are involved in reprogramming, was increased in the presence of a small molecule that functions to inhibit histone deacetylases.

TABLE VI
Change in expression of pluripotency genes in the presence of
VPA

| | Oct4 | | Nanog | | Sox2 | | HDAC11 | |
|---|---|---|---|---|---|---|---|---|
| Cell | (Fold | | (Fold | | (Fold | | (Fold | |
| type | increase) | P | increase) | P | increase) | P | increase) | P |
| HDFa | 3.40 | <0.01 | 3.82 | <0.01 | 3.60 | <0.01 | 2.96 | <0.01 |
| HDFf | 5.27 | <0.01 | 8.09 | <0.02 | 1.11 | <0.03 | 4.07 | <0.001 |
| HDFn | 3.66 | <0.03 | 2.01 | <0.03 | 4.45 | <0.05 | | |
| BJF | 7.29 | <0.01 | 6.64 | <0.01 | 8.77 | <0.02 | | |

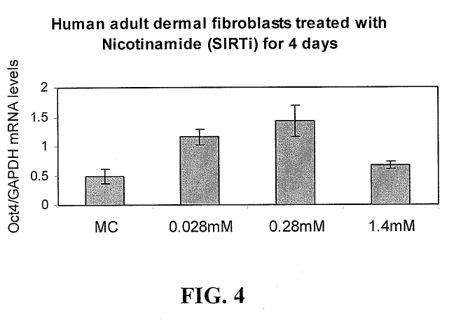

As shown in FIG. 4, Oct-4 expression was increased when human adult dermal fibroblasts were treated with nicotinamide for four days. All three concentrations tested, 0.028 mM, 0.28 mM, and 1.4 mM, led to an increase in Oct-4 expression as compared to the control medium (MC). These data demonstrate that a small molecule inhibitor, in this case nioctinamide, increase the expression of Oct-4, which is a gene involved in reprogramming a differentiated cell and restoring differentiation potential to a cell.

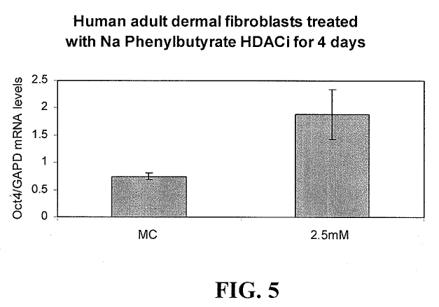

As shown in FIG. 5, Oct-4 expression was increased when human adult dermal fibroblasts were treated with sodium phenylbutyrate. The cells were treated for four days in the presence of sodium phenylbutyrate (2.5 mM). As compared to the control medium (MC), the treated cells demonstrated an increase in Oct-4 expression. These data demonstrate that small molecule inhibitors that function to inhibit histone deacetylases can be used to increase the expression of a pluripotent or multipotent gene and reprogram a cell.

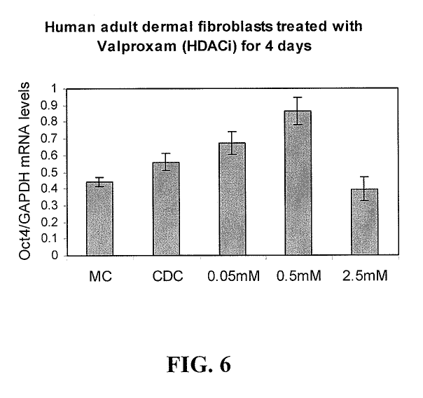

As shown in FIG. 6, the expression of a pluripotent gene, Oct-4, was increased when human adult dermal fibroblasts were treated with valproxam for four days. At two concentrations of valproxam, 0.05 mM, and 0.5 mM, Oct-4 expression was increased when compared to the control medium (MC). At 2.5 mM valproxam, Oct-4 expression appeared to return to baseline level (similar level to the control medium). This may reflect a decrease in the number of viable cells, or may be indicative of an experimental limitation imposed by the equipment.

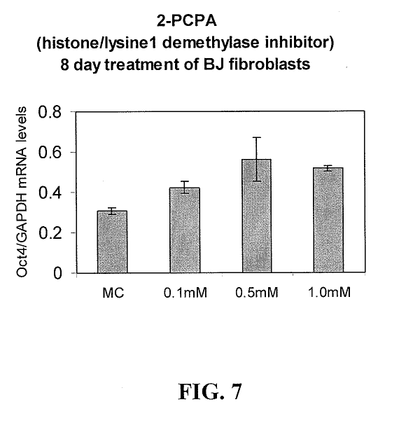

As shown in FIG. 7, Oct-4 expression was increased when BJ fibroblasts were treated 2-PCPA for eight days. The compound 2-PCPA is a histone/lysine 1 demethylase inhibitor. All three concentrations of 2-PCPA, 0.1 mM, 0.5 mM, and 1.0 mM, resulted in an increase in the expression of Oct-4 as compared to the control medium (MC).

Small molecule modulators, targeted toward multiple targets, including but not limited to histone deacetylases, and SIRTs, can be used to increase the expression of pluripotent or mulitpotent genes, and can be used to reprogram a differentiated cell. These reprogramming methods are independent of eggs, embryos or embryonic stem cells. Furthermore, these methods do not rely on viral vectors, which can have harmful effects. These methods are also independent of oncogenes, such as c-myc and Klf4.

In addition, the methods of the present invention can be used to reprogram a differentiated cell in the absence of somatic cell nuclear transfer (SCNT). SCNT is very inefficient and has posed a significant limitation on the field of reprogramming. The present methods alleviate the need for SCNT.

The present methods have demonstrated an increase in expression of the endogenous Oct-4 gene, as opposed to an artificial vector with a strong reporter element. An artificial vector does not have the same chromatin structure as the endogenous gene, nor does it have other genes, and promoter elements to create the environment of the genome. An artificial vector does not have many of the natural elements needed to recapitulate the environment of the natural genome. The results presented herein represent effects obtained from treating human cells, and measuring the effects on the endogenous gene.

Finally, the data presented herein demonstrate that small molecule modulators can be used to alter the function of protein complexes, such as histone deacetylases. Altering chromatin structure is a step in reprogramming a differentiated cell, and restoring differentiation potential.

### Example 3

The morphological changes induced by exposure to VPA were examined. Embryonic cells have distinct morphological characteristics. Therefore, cells treated with VPA were examined to determine if the increase in expression of pluripotency genes correlated with morphological changes consistent with embryonic cells.

Methods

Human adult dermal fibroblasts were treated with 500 μM VPA in fibroblast growth medium for 5 days in 24-well plates. The cells were re-treated with 500 μM VPA on day 3. At the end of five days, cells were then transferred to 6-well plates and treated daily with 500 μM VPA in mTeSR hES cell culture medium (available from StemCell Technologies, Vancouver, BC, Canada) for an additional 16 days; mTeSR medium was changed daily. When colonies were observed in suspension, at approximately day 21, the cells were transferred to matrigel plates and photographed after plating.

Results

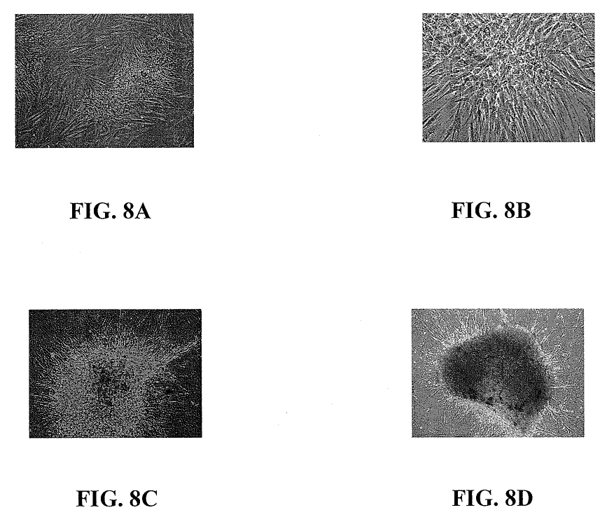

FIGS. 8A-8D are photographs of untreated cells and cells treated with 500 μM VPA. FIG. 8A is a photograph of untreated cells in fibroblast growth medium. FIG. 8B is a photograph of untreated cells in DMEM/F12 medium. FIG. 8C and FIG. 8D are photographs of VPA-treated cells in mTeSR hES cell medium on matrigel. Cells treated with VPA resemble embryoid-like colonies (FIG. 8C) and embryoid-like bodies (FIG. 8D). However, no positive pluripotent protein staining was detected (data not shown). This could be the result of experimental error or a limitation on the experimental system.

Cells treated with a small molecule modulator induced expression of genes, such as Oct-4 and Nanog, which are two genes involved in maintaining the pluripotential of a cell, and involved in reprogramming a differentiated cell. In addition, increasing the expression of these genes resulted in morphological changes in the cells, wherein the morphological changes were consistent with embryonic-like cells. These results clearly suggest that small molecule modulators, such as an inhibitor of histone deacetylases, can be used to transform a differentiated cell into an embryonic-like cell. The results support the notion that cells can be reprogrammed by exposing the cells to a small molecule inhibitor, such as VPA.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof. The disclosures of patents, references and publications cited in the application are incorporated by reference herein.

What is claimed is:

1. A method for reprogramming a cell comprising: exposing a cell to a small molecule modulator that induces expression of a pluripotent gene; and selecting a cell, wherein differentiation potential has been restored to said cell.

2. The method of claim 1, wherein selecting said cell comprises: comparing phenotypes of the cell prior to and after exposure to said small molecule modulator, and identifying a cell with a phenotype consistent with restored differentiation potential.

3. The method of claim 1, further comprising: expanding the selected cell to a population of cells.

4. The method of claim 1, wherein the small molecule modulator is selected from the group consisting of: a histone deacetylase inhibitor, a methyl binding domain protein inhibitor, a methyl adenosyltransferase inhibitor, a DNA methyltransferase inhibitor, a histone methyltransferase inhibitor, a lysine methyltransferase inhibitor, a histone demethylase inhibitor, and a methyl cycle enzyme inhibitor.

5. The method of claim 4, wherein said small molecule modulator is a DNA methyltransferase inhibitor.

6. The method of claim 5, wherein said DNA methyltransferase inhibitor is RG108.

7. The method of claim 1, wherein selecting said cell comprises: isolating a cell using an antibody directed to a protein expressed from a pluripotent gene or a pluripotent marker.

8. The method of claim 1, wherein selecting said cell comprises: isolating a cell using a reporter driven by a pluripotent gene or resistance to a selectable marker.

9. The method of claim 1, wherein said pluripotent gene is selected from the group consisting of: Oct-4, Sox-2, and Nanog.

10. The method of claim 1 further comprising: comparing chromatin structure of a pluripotent gene of said cell prior to exposure to said small molecule modulator to the chromatin structure obtained after exposure to said small molecule modulator.

11. A method for reprogramming a cell comprising: exposing a cell with a first transcriptional pattern to a small molecule modulator, wherein said modulator induces expression of a pluripotent gene; comparing the first transcriptional pattern of the cell to a transcriptional pattern obtained after exposure to said modulator; and selecting a cell, wherein differentiation potential has been restored to said cell.

12. The method of claim 11, wherein said transcriptional pattern after exposure to said modulator is at least 50% similar to the transcriptional pattern of an embryonic stem cell.

13. The method of claim 11, wherein prior to comparing the transcriptional patterns, phenotypes of the cell prior to and after exposure to said small molecule modulator are compared.

14. The method of claim 11, further comprising: expanding the selected cell to a population of cells.

15. The method of claim 11, wherein selecting said cell comprises: isolating cells using an antibody directed to a protein expressed from a pluripotent gene or a pluripotent marker.

16. The method of claim 11, wherein said pluripotent gene is selected from the group consisting of: Oct-4, Sox-2, and Nanog.

17. An enriched population of reprogrammed cells produced according to a method comprising the steps of: exposing a cell to a small molecule modulator that induces expression of a pluripotent gene; and selecting a cell, wherein differentiation potential has been restored to said cell, and culturing said selected cell to produce a population of cells.

18. The enriched population of reprogrammed cells of claim 17, wherein the reprogrammed cell expresses a cell surface marker selected from the group consisting of:
SSEA3, SSEA4, Tra-1-60, and Tra-1-81.

19. The enriched population of reprogrammed cells of claim 17, wherein the pluripotent gene is selected from the group consisting of: Oct-4, Nanog, and Sox-2.

20. The enriched population of reprogrammed cells of claim 17, wherein said reprogrammed cells account for at least 60% of the population.
